(19) United States
(12) Patent Application Publication
(10) Pub. No.: US 2004/0071715 A1
(43) Pub. Date: Apr. 15, 2004

(54) Polymer compositions that stabilize and control the release of formaldehyde-treated vaccine antigens

(76) Inventors: Steven P. Schwendeman, Ann Arbor, MI (US); Wenlei Jiang, Bridgewater, NJ (US)

Correspondence Address: BROOKS KUSHMAN P.C., 1000 TOWN CENTER, TWENTY-SECOND FLOOR, SOUTHFIELD, MI, 48075, US

(21) Appl. No.: 10/417,841

(22) Filed: Apr. 17, 2003

Related U.S. Application Data
(63) Continuation-in-part of application No. 09/738,961, filed on Dec. 15, 2000.
(60) Provisional application No. 60/170,983, filed on Dec. 15, 1999.
(60) Provisional application No. 60/373,858, filed on Apr. 19, 2002.

(51) Int. Cl. A61K 39/00; A61K 9/14

(52) U.S. Cl. 424/184.1; 424/486

(57) ABSTRACT

A delivery system and a method for sustained release of formaldehyde-treated vaccine antigens wherein the antigens are stabilized by stabilizing agents mixed with the formaldehyde-treated antigens and then dispersed within a biocompatible polymer matrix. The stabilizing agents inhibit formaldehyde mediated aggregation of formaldehyde-treated vaccine antigens by greater than 60% compared to antigens incubated without stabilizing agents. Stabilizing agents may be selected from formaldehyde-interacting amino acids, basic additives, and mono, di-, or polysaccharides. The delivery system may be biodegradable. The formaldehyde-treated vaccine antigens my be tetanus toxoid, diphtheria toxoid, or both tetanus toxoid and diphtheria toxoid.

### CROSS-REFERENCE TO RELATED APPLICATIONS

This application claims the benefit of U.S. Provisional Application Serial No. 60/373,858 filed, Apr. 19, 2002. That application is incorporated by reference herein.

### BACKGROUND OF THE INVENTION

1. Field of the Invention

This invention relates to a biocompatible polymer delivery system and method that stabilizes and controls the release of formaldehyde-treated vaccine antigens.

2. Background Art

Biocompatible polymers have been investigated to provide controlled release from days to months for therapeutic proteins and vaccine antigens (Schwendeman et al., In Controlled Drug Delivery, Challenges and Strategies. The American Chemical Society: 229-267, 1997). Copolymers derived from lactic and glycolic acids, i.e. poly(lactide-co-glycolide) (PLGA), when formed into microspheres have shown great potential as carriers for single-dose vaccine delivery, and the advantages of PLGA microspheres are well known (Hanes et al., Adv. Drug Del. Rev. 28: 97-119, 1997; Schwendeman et al., In Controlled Drug Delivery, Challenges and Strategies. The American Chemical Society: 229-267, 1997; Gupta et al., Dev. Biol. Stand. 92: 63-78, 1998; Gupta et al., Adv. Drug Del. Rev. 32: 225-246, 1998).

Incomplete release and poor stability of encapsulated proteins are common hurdles to overcome when developing PLGA controlled release systems. One major obstacle in developing a biocompatible polymer system for controlled release is the instability of encapsulated protein antigens during exposure to physiological conditions, i.e., elevated humidity and temperature over time. Protein antigens may undergo hydrolysis, oxidation, deamidation, aggregation, as well as other deleterious processes in polymer release systems, resulting in the loss of antigenicity or incomplete release of active antigens (Schwendeman et al., In Controlled Drug Delivery, Challenges and Strategies. The American Chemical Society: 229-267, 1997; O'Hagan et al., Adv. Drug Del. Rev. 32: 225-246, 1998). For adequate delivery over a period of days up to months, vaccine antigens must remain encapsulated at physiological temperature, which poses significant challenges regarding retention of both the structural integrity and biological activity of the protein antigens (Schwendeman et al., In Microparticulate Systems for the Delivery of Proteins and Peptides, 1-49, 1996).

The release and stability of protein antigens in PLGA systems is based on several factors. Protein antigen stability is related to the physical and chemical properties of antigens themselves and also the physical and chemical processes during microsphere preparation, storage and in vivo antigen release (Schwendeman et al., In Controlled Drug Delivery, Challenges and Strategies. The American Chemical Society: 229-267, 1997). Protein release from biodegradable polymers is controlled by a combination of protein diffusion through aqueous pores, polymer erosion, and osmotically mediated events. Very often, the more water the polymer matrix absorbs, the more rapid the protein release. The importance of the water uptake kinetics during the release is also emphasized by two other well-known phenomena: (1) protein stability is related to water content (Hageman, In Stability of Protein Pharmaceuticals, 273-309,1992; Akbari et al., Pharm. Dev. Technol. 3: 251-259, 1998); e.g. the moisture-induced degradation/aggregation is believed to be one of the major sources of the incomplete release of protein from polymer delivery systems (Schwendeman et al., In Controlled Drug Delivery, Challenges and Strategies. The American Chemical Society: 229-267, 1997); and (2) water content typically affects the microenvironment inside the polymer by diluting, and accelerating the release of, encapsulated agents and soluble monomers/oligomers produced accompanying degradation of the polymer (Akbari et al., Pharm. Dev. Technol. 3: 251-259, 1998).

Another factor affecting the release and stability of proteins in the PLGA delivery system is the common formation of an acidic microclimate within the polymer device during release due to polymer degradation, which leads to destablilzation of encapsulated acid-labile bio-macromolecules. Previous work has demonstrated that the acidic microclimate and intermediate water content existing in PLGA 50/50 delivery systems were the major causes of instability of encapsulated proteins (i.e. bovine serum albumin, (BSA)). Antacid agents, which increase both microclimate pH and effect polymer water uptake, have been shown to reduce acid induced instability of proteins encapsulated in PLGA (Zhu et al., Nat. Biotechnol. 18(1): 52-57, 2000, Zhu and Schwendeman, Pharm. Res. 17(3): 351-357, 2000).

Another obstacle in developing controlled release delivery systems occurs when protein antigens acquire properties from their preparation procedures, which may cause some additional instability problems. Formaldehyde treatment is one method of protein antigen preparation that has been used to convert toxins into relatively inert but still antigenic toxoids. Formaldehyde-treated antigens have been shown to possess special reactive species that trigger a unique formaldehyde-mediated aggregation at intermediate moisture levels (Schwendeman et al., Proc. Natl. Acad. Sci. USA 92: 11234-11238, 1995; Jiang and Schwendeman, Biotech. Bioeng. 70(5): 507-517, 2000).

Through a series of reactions, formaldehyde treatment of antigens results in the formation of nondisulfide-linked covalently bonded insoluble aggregates. Briefly, labile antigen-formaldehyde linkages form during formaldehyde treatment and are converted to highly reactive electrophiles (e.g. Schiff bases) under appropriate conditions. When lyophilized antigens are incubated at elevated humidity and temperature, Schiff bases may combine with nucleophiles within the same or different protein molecules to form intra- and intermolecular crosslinks. In the latter case, this leads to the formation of nondisulfide-linked covalently bonded insoluble aggregates. This process has been termed the formaldehyde-mediated aggregation pathway (FMAP). The protein aggregates formed during formaldehyde treatment of vaccine antigens prevent controlled release of the antigen and may adversely affect the desired immune response. (Jiang and Schwendeman, J. Pharm. Sci. 90(10): 1558-1569, 2001).

A central hypothesis for the success of a single-dose antigen vaccine include the necessity for uninhibited antigen release from the biocompatible polymer delivery system and that the released antigen has not undergone extensive aggregation, degradation, or denaturation, allowing the antigen to remain competent as an immunogen to produce antibodies recognizing and neutralizing toxins. A need exists for development of a biocompatible polymeric delivery system capable of sustained, controlled release of formaldehyde detoxified vaccine antigens wherein the problems of antigen release instability, aggregation, and loss of antigenicity in polymeric systems have been solved.

### SUMMARY OF THE INVENTION

The present invention relates to a delivery system for sustained release of a formaldehyde-treated vaccine antigen from a biocompatible polymer matrix which stabilizes and controls the release of the formaldehyde-treated vaccine antigen and preserves antigenicity during release and delivery. The delivery system comprises a biocompatible polymer matrix, at least one formaldehyde-treated vaccine antigen, and at least one stabilizing agent. The stabilizing agents stabilize the formaldehyde-treated vaccine antigen and inhibit the formation of aggregates when the stabilizing agent is encapsulated with the formaldehyde-treated vaccine antigen within the biocompatible polymer matrix.

Stabilizing agents sufficient to inhibit formation of aggregates of formaldehyde-treated BSA by greater than 60% in the bovine serum solubility assay are preferred for inclusion in the biocompatible polymer delivery systems for formaldehyde-treated vaccine antigens. Additional agents further enhance the stability and controlled release of the delivery system, including formaldehyde-interacting amino acids, basic stabilizing additives, and mono- and disaccharides.

The present invention also relates to a method for stabilizing formaldehyde-treated vaccine antigens for controlled release in a biocompatible polymeric delivery system by forming a biocompatible polymer matrix, encapsulating the formaldehyde-treated vaccine antigen and at least one stabilizing agent in the biocompatible polymer matrix and solidifying the biocompatible polymer matrix, the formaldehyde-treated vaccine antigen, and at least one stabilizing agent to provide a biocompatible polymeric delivery system for controlled release of formaldehyde-treated vaccine antigens.

The above objects and other objects, features, and advantages of the present invention are readily apparent from the following detailed description of the best mode for carrying out the invention when taken in connection with the accompanying drawings.

### BRIEF DESCRIPTION OF THE DRAWINGS

FIG. 1 is a graph of cumulative release kinetics of f-BSA from f-BSA/PLA (i.v.=1.07 dl/g) O/O microspheres (▪), f-BSA/PLA (i.v.=1.07 dl/g)/PEG35,000 O/O microspheres (▴), f-BSA+histidine+trehalose/PLA (i.v.=1.07)/PEG35,000 O/O microspheres (●) at 37° C. (Average±s.d., n=3);

FIG. 2A is a graph of release kinetics of tetanus toxoid from PLGA 85/15 (i.v.=0.86 dl/g) microspheres co-encapsulated with lysine+MgCO₃; Protein content (●) and antigenic tetanus toxoid (∘) released from microsphere size <120 μm when release medium contains no BSA; Antigenic TT (□) released from microsphere size <120 μm when release medium contains 0.2% BSA;

FIG. 2B is a graph of release kinetics of tetanus toxoid from PLGA 85/15 microspheres co-encapsulated with lysine+sorbitol+MgCO₃. Protein content (●) and antigenic tetanus toxoid (∘) released from microsphere size <120 μm when release medium contains no BSA; Antigenic TT (□) released from microsphere size <120 μm when release medium contains 0.2% BSA;

FIG. 2C is a graph of release kinetics of tetanus toxoid from PLGA 85/15 microspheres co-encapsulated with lysine+trehalose+MgCO₃; Protein content (●) and antigenic tetanus toxoid (∘) released from microsphere size <120 μm when release medium contains no BSA; Antigenic TT (□) released from microsphere size <120 μm when release medium contains 0.2% BSA.

FIG. 3A is a scanning electron micrograph of tetanus toxoid O/O encapsulated in PLGA 85/15 (0.86 dl/g) microspheres containing lysine, sorbitol, and MgCO₃ before incubation;

FIG. 3B is a scanning electron micrograph of tetanus toxoid encapsulated in PLGA 85/15 microspheres containing lysine, sorbitol, and MgCO₃ after 28 days incubation;

FIG. 4A is a scanning electron micrograph of PLA (i.v.=1.07 dl/g) microspheres before and after incubation in release medium;

FIG. 4B is a scanning electron micrograph of PLA/PEG 35,000 (90/10) microspheres before and after incubation in release medium;

FIG. 4C is a scanning electron micrograph of PLA/PEG 35,000 (80/20) microspheres before and after incubation in release medium;

FIG. 5A is a graph of the effect of PEG content and molecular weight in the PLA/PEG blend on the release kinetics of BSA. PEG 10,000 content was 0% (●), 5% (▪), 10% (▴), and 20% (▾) (Average±s.d., n=3);

FIG. 5B is a graph of the effect of PEG content and molecular weight in the PLA/PEG blend on the release kinetics of BSA. PEG molecular weight and content were 20% PEG 10,000 (▪) and 20% PEG 35,000 (●), 30% PEG 35,000 (▴) (Average±s.d., n=3).

### DETAILED DESCRIPTION OF THE PREFERRED EMBODIMENT(S)

The present invention provides a delivery system for controlled release of formaldehyde-treated vaccine antigens from biocompatible polymers.

Biocompatible polymers are defined as polymers and any degradation products of the polymers that are substantially non-toxic and non-immunogenic to the recipient. Biocompatible polymers do not illicit untoward physiologic responses, or do so only very infrequently, thereby avoiding detrimental effects of the body's protective systems and remaining functional for a significant period of time. Many such biocompatible polymers are known and have received approval by regulatory agencies in this and other countries for pharmaceutical use.

Polymeric delivery systems can be subcategorized into non-degradable and biodegradable polymer systems. Non-degradable polymers as defined herein do not degrade under physiological conditions, such as by hydrolysis or enzymatic attack. The non-degradable polymers used herein are biocompatible polymers such as polyacrylates, polymers of ethylene-vinyl acetates, acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof. Copolymers from ethylene and vinyl acetate and organopolysiloxanes are preferred non-degradable biocompatible polymers.

Biodegradable polymers are defined herein as polymers that are degraded to form smaller chemical species under physiological conditions. Potential biocompatible, biodegradable polymers include poly(lactide)s, poly(glycolides)s, poly(lactide-co-glycolide)s, polyanhydrides, polyorthoesters, polyetheresters, polycaprolactones, polyesteramides, polyoxalates, and blends and copolymers thereof. The family of biodegradable homo-and co-polymers derived from lactic and glycolic acid-poly(lactic acid) (PLA) or poly (lactic-co-glycolic acid) (PLGA) are preferred for controlled release vaccine antigen delivery systems. Poly (lactic-co-glycolic acid) (PLGA) polymers are preferred for preparing biocompatible delivery systems for formaldehyde-treated vaccine antigens because of their safety, FDA approval, and biodegradability.

Formaldehyde treatment has been used to convert toxins into relatively inert but still antigenic toxoids since the early 1900s (Lowenstein, Z Hyg Infecktionskrankh 62: 491-508, 1909). Any method of formaldehyde treatment known in the art is acceptable for treating vaccine antigens wherein the antigenicity of the resulting toxoid is not seriously compromised.

By organic formaldehyde-interacting stabilizing agent is meant an organic compound, natural or synthetic, which decreases aggregation of formaldehyde-treated vaccine antigens as measured by at least a 60% retention of unaggregated antigen after storage under physiological conditions, i.e. 37° C., 80% humidity, 30 days as disclosed in detail herein. The stabilizing agents preferably exhibit a greater than 80% retention of unaggregated antigen under these conditions, more preferably greater than 90% .

If two or more organic formaldehyde-interacting stabilizing agents are employed, it is the combination which is tested for retention of unaggregated antigens and not the individual agents. The test is conducted with the same antigen in the same polymer matrix, but without moderately basic inorganic additions. Alternatively, formaldehyde-treated bovine serum albumin may be used in place of the antigen to test the stabilizing agents.

Preferred organic stabilizing agents are identified elsewhere herein. Suitable groups of stabilizing agents include oligopeptides, particularly those rich in lysine, arginine, and glutamine, and sugars, and aminosugars, including mono-, di- and oligosaccharides. The suitability of a particular stabilizing agent is easily assessed by those skilled in the art.

Polymeric delivery systems may be processed into miniature devices such as, but not limited to, microspheres, nanospheres, microcapsules, and cylindrical rods. Delivery of the formaldehyde-fixed vaccine antigen may also be accomplished with in-situ forming polymer melts or polymer solutions. To mimic the particulate nature of pathogens and considering the convenience and safety of administration, PLGA microspheres have been consequently preferred in vaccine antigen delivery.

Administration of the polymeric delivery systems may be through injection, inhalation, implantation, transdermal, mucosal, and oral routes.

Controlled release of formaldehyde-treated vaccine antigens allows release of immunocompetent antigens over a prolonged period of time while maintaining the stability, solubility, and antigenicity of the antigens during degradation of the polymeric delivery system. Controlled release of antigens may also occur by diffusion, or osmotically mediated events from a non-degradable or a biodegradable polymeric system. Controlled release of immunocompetent formaldehyde-treated vaccine antigens occurs over a period of days to more than one year, preferably at least four weeks.

In a preferred embodiment, formaldehyde-treated vaccine antigens are released from biocompatible polymer delivery systems. Stabilizing agents are combined with the formaldehyde-treated vaccine antigen and then encapsulated within the biocompatible polymer matrix. The stabilizing agents inhibit the formaldehyde mediated aggregation of the antigen in a humid environment and at a physiological temperature, resulting in minimizing solubility and antigenicity losses of the encapsulated antigen. Preferably, the controlled release occurs from days up to months or longer, providing vaccine antigens competent to elicit an immunogenic response to produce antibodies recognizing and neutralizing toxins which are effective for extended periods of time.

Stabilizing agents suitable for use in inhibiting formaldehyde mediated aggregation of vaccine antigens may be determined by using an assay to assess the ability of the agent to inhibit the aggregation of formaldehyde-treated bovine serum albumin (f-BSA) when cultured for 4 days under physiologic conditions expected to trigger formaldehyde mediated aggregation, namely 80% relative humidity (RH) and 37° C. Solubility of the f-BSA after co-lyophilization of f-BSA with the test agent and subsequent rehydration in water is measured by the Coomassie Plus method (Pierce Chemical Co.) or by the optical density of the proteins at 280 nm. (Bovine serum albumin solubility assay.) The agent is sufficiently stabilizing for the formaldehyde-treated vaccine antigen delivery system when the agent inhibits formation of aggregates by 60% or greater in the bovine serum albumin solubility assay.

Amino acids may be used as stabilizing agents to inhibit formaldehyde-treatment induced aggregation of vaccine antigens. Both α-amino groups and nucleophilic groups of side chains in amino acids are capable of attacking reactive electrophiles and blocking the formation of crosslinks among protein molecules. Amino acids considered effective inhibitors of aggregation in the FMAP pathway for use as stabilizing agents were determined by the ability of specific amino acids to inhibit the formation of aggregates of formaldehyde treated bovine serum albumin (f-BSA). Amino acids capable of inhibiting aggregation of f-BSA by greater than 60% are preferred for use as stabilizing agents. Preferred amino acids are histidine, lysine, arginine, and glutamine.

Additional potential stabilizing agents may be tested in the bovine serum solubility assay, such as, but not limited to, D-amino acids and peptides. The formaldehyde-treated vaccine antigen itself may be substituted for the f-BSA in the solubility assay to select sufficiently stabilizing agents specific to each formaldehyde-treated vaccine antigen. Preferred agents stabilize release of formaldehyde-treated vaccine antigens for days up to a year, more preferably for minimally four weeks.

In another embodiment, in addition to a stabilizing agent, a method for preparing a delivery system which stabilizes the formaldehyde-treated vaccine antigen encapsulated therein during biodegradation comprises additionally adding a poorly soluble, basic additive to a solution comprising the formaldehyde-treated vaccine antigen and the polymer. Acidic species are produced during bioerosion of the polymer and addition of a basic additive further stabilizes the formaldehyde-treated vaccine antigen. Except for Ca(OH)₂, the basic additive may, for example, have a solubility and basicity comparable to the compounds shown in Table 1 below.

TABLE 1
Solubility and basicity of basic salts.

| | | | pH of | |
|---|---|---|---|---|
| | | | saturated | Addition of 100 |
| Salts | pKₛₚ^{a} | Solubility^{b} | solution^{c} | μl of 1 N HCl^{d} |
| Ca(OH)₂ | 5.26 | 1.11 × 10⁻² | 12.40 | 12.20 |
| CaCO₃ | 8.42 | 6.17 × 10⁻⁵ | 9.26 | 6.07 |
| Ca₃(PO₄)₂ | 26.0 | 3.12 × 10⁻¹¹ | 7.77 | 3.71 |
| Mg(OH)₂ | 10.74 | 1.66 × 10⁻⁴ | 9.76 | 8.99 |
| MgCO₃ | 5.00 | 3.16 × 10⁻³ | 9.75 | 9.01 |
| Zn(OH)₂ | 15.68 | 3.74 × 10⁻⁶ | 8.85 | 5.86 |
| ZnCO₃ | 10.78 | 4.07 × 10⁻⁶ | 7.34 | 5.36 |
| Zn₃(PO₄)₂ | 32.0 | 1.24 × 10⁻¹³ | 6.82 | 1.53 |

^{a}Lange's Handbook of Chemistry, Ed. John A. Dean, 11^{th} edition, 1973.
^{b}Solubility was calculated based on pKₛₚ values;
^{c}pH was measured after excess basic salts were suspended in 10 ml of water and incubation was continued at 37° C. for 7 days;
^{d}pH was measured after the acid was added to the above suspension and incubation was continued at 37° C. for 3 days.

Further examples of suitable basic additives are magnesium carbonate, magnesium hydroxide, magnesium oxide, magnesium trisilicate, zinc carbonate, zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, and magaldrate.

Preferably, the polymer comprises from 50% to 100% lactide or lactic acid, which may be a D- isomer, L-isomer, or a D-,L-racemic mixture, and from 50% to 0% of a glycolide or glycolic acid. The polymer may have an inherent viscosity of from 0.1 to 2.0 dl/g, but any degree of polymerization which allows formulation of the specific biocompatible polymer product is satisfactory.

The polymer solution may comprise from 0.5 to 20% of the basic additive. In those cases where the amount of basic additive dispersed in the solution is low, i.e. from 0.5% to 3% w/w, it is preferred that the porosity of the polymeric delivery system be increased. Methods for increasing the porosity of the polymeric delivery system include adding a pore-forming agent to the polymer solution, increasing the amount of formaldehyde-treated vaccine antigen or the composition comprising the formaldehyde-treated vaccine antigen and carrier to a value of 5 to 20% (w/w), or using a low concentration of polymer, e.g. 40-300 mg/ml of polymer in the organic solvent. In those cases where the polymer concentration is high, e.g. 1200 mg/ml or the inherent viscosity is high, it is preferred that the polymer solution comprise from 3 to 20% by weight of the basic additive.

A further method for preparing enhanced biodegradable polymeric delivery systems involves blending a pore-forming agent with a polymer which comprises from 50% to 100% lactide or lactic acid and from 50% to 0% glycolide or glycolic acid. Examples of suitable pore forming agents are polyethylene glycol (PEG) and water soluble polyoxamers. Preferably, the pore-forming agent has a molecular weight of from 500 to 30,000, more preferably from 4000 to 10,000. Additional pore forming agents may be selected from those know to the artisan skilled in preparing microporous membranes where use of pore-forming ingredients is commonplace.

In addition to inclusion of stabilizing agents, polymeric systems may be designed to minimize acid build-up found in PLGA polymers to further stabilize the formaldehyde-treated antigens for controlled release. A blend of slowly degrading poly (D, L-lactide) (PLA), to reduce the production of acidic species during protein release, and water-soluble poly (ethylene glycol)(PEG), to increase diffusion of the protein and polymer degradation products, have been found to modify the microsphere microclimate and protein release behavior. PLA has a much slower degradation rate than PLGA 50/50 due to its higher hydrophobicity and the stearic hindrance against attack by water on the ester bond introduced by the methyl group of lactic acid (Magre and Sam, J. Control Rel. 48: 318-319, 1997). Likewise, PLA has been shown to produce acidic by-products and release monomers at a reduced rate relative to the glycolic-containing copolymers (Shenderova, Ph.D. Thesis. The Ohio State University, 2000; Giunchedi et al., J. Control Rel. 56: 53-92, 1998). Like PLGA 50/50, PLA will still cause an eventual acid build-up. In addition, its slow degradation may lead to slow and discontinuous release of proteins.

In addition, the strong hydrophobicity of PLA has been suggested to possibly denature proteins (Crotts and Park, J. Microencapsul. 15: 699-713, 1997). Therefore, the second component, relatively more hydrophilic PEG, may be introduced into PLA to adjust the microsphere acidity, hydrophobicity, and permeability. PEG is nontoxic and soluble in numerous organic solvents and water. During release PEG dissolves in and diffuses into the release medium, resulting in the formation of a swollen structure with high water content in the polymer blend. This swollen polymer structure increases exchange of polymer degradation products with the surrounding medium, minimizing the risk of acid-induced protein degradation. Moreover, before excessive PLA degradation occurs, hydrophilic pores formed by PEG dissolution also increase diffusion of the encapsulated protein, providing continuous protein release.

The PLA-PEG microspheres studied here were prepared by an oil-in-oil emulsion and solvent extraction (o/o) method. The (o/o) method generally results in high protein entrapment levels and superior protein stability due to the absence of water. A model protein antigen, bovine serum albumin (BSA), was selected and encapsulated in the polymer blend. Stabilization and slow release of BSA from PLA-PEG microspheres occurred without any observed acid-induced instability. Table 2 shows the results of the effect of PEG on BSA aggregation.

TABLE 2
The effect of PEG on BSA aggregation in o/o microspheres after 29-day
release study in PBST at 37° C.

| | | | | | | | | | Non- | | | |
|---|---|---|---|---|---|---|---|---|---|---|---|---|
| | | | | | | | | | covalent | | | |
| | | | | | | | | | and | | | |
| | | | | | | | Non- | | disulfide | | | |
| | MW of | Wt | | | | | covalent | | bonded | | | |
| Form | PEG in | % of | Released | | Soluble | | aggregates | | aggregates | | | |
| Code | blend¹ | PEG | BSA % | | BSA %² | | %³ | | %⁴ | | Recovery % | |
| o | — | 0 | 36 ± | 1⁵ | 15 ± | 1⁵ | 22 ± | 1⁵ | 25 ± | 2⁵ | 76 ± | 2⁵ |
| a | 10 kD | 5 | 44.6 ± | 0.3 | 30 ± | 2 | 41 ± | 2 | 41 ± | 7 | 116 ± | 7 |
| b | 10 kD | 10 | 40.4 ± | 0.3 | 39 ± | 2 | 26 ± | 4 | 36 ± | 4 | 115 ± | 4 |

| | | | | | | | | | | |
|---|---|---|---|---|---|---|---|---|---|---|
| c | 10 kD | 20 | 73.2 ± | 0.3 | 37 ± | 4 | — | — | 110 ± | 4 |
| d | 35 kD | 20 | 75.5 ± | 0.3 | 30.2 ± | 0.2 | — | — | 106 ± | 1 |
| e | 35 kD | 30 | 82.3 ± | 0.2 | 30 ± | 2 | — | — | 112 ± | 2 |

¹Theoretical protein loading was 5%; PLA (i.v. = 1.07 dl/g) was used in the formulation and the total polymer concentration (w/v) was 20%.
²Soluble in PBST.
³Soluble in denaturing agents (8 M Urea).
⁴Soluble in combined denaturing and reducing agents (8 M Urea and 10 MM DTT).
⁵The BSA release study from the PLA specimen was performed over 45 days and the residue was recovered after this time.

Stabilization of formaldehyde-treated vaccine antigens is also dependent on the method of encapsulation of the antigens within the polymer matrix. Among the microencapsulation techniques, the water-in-oil-in-water double emulsion-solvent evaporation method (w/o/w) is one of the most convenient ways to encapsulate proteins in PLGA microspheres. However, the presence of the aqueous phase may increase the diffusion of protein to the external phase, resulting in the decreased encapsulation efficiency, especially when high protein loading is required. Furthermore, proteins in general become more labile when confronting the organic solvent-water interface. Compared with the w/o/w method, the oil-in-oil single emulsion-solvent extraction method (o/o) system is preferred for antigen stability. A comparison of the effect of the encapsulation method on the stability of f-BSA is shown in Table 3.

TABLE 3
Effect of encapsulation method.

| | Incubation | Soluble f-BSA |
|---|---|---|
| Antigen Formulations | Condition | (%)^{b} |
| Effect of | | |
| encapsulation method | | |
| f-BSA w/o/w microspheres^{a} | 97% RH, 37° C., 16 days | 50 ± 2 |
| f-BSA o/o microspheres^{a} | 97% RH, 37° C., 16 days | 112 ± 1 |

^{a}Theoretical protein loading was 8%; PLGA50/50 (iv = 0.64 dL/g) was used for microsphere preparation.
^{b}Soluble in PBST; data represent average ± SD, n = 3.

In another embodiment, a stabilizing agent may be a mono- or disaccharide. A study was performed to target the specific formaldehyde-mediated reactivity intrinsic to formalized antigens. A series of sugars, including sucrose, trehalose, sorbitol, mannitol, and dextrose, were tested for their impact on the stability of lyophilized f-BSA. These sugars were co-lyophilized with f-BSA separately as a weight ratio of 1:5 (agent:protein) and then protein powders were incubated at 37° C. and 80% RH. After 14 days of incubation, it was shown that co-incorporation of either sorbitol or trehalose was most effective, largely increasing the soluble f-BSA percentage to 33% and 29%, respectively, from 9% (without additive). Sucrose, mannitol, and fructose increased the soluble f-BSA percentage to 18%, 20%, and 15% respectively.

Amino acids and sugars were also tested to evaluate their effect on the antigenic functional groups of tetanus toxoid. Agents in solution with tetanus toxoid were exposed to heat at 45° C. for 22 days to analyze heat-induced antigen instability. Results are shown in Table 4. Additionally, samples of agents mixed with tetanus toxoid and colyophylized were incubated at 37° C. and 80% RH for 15 or 32 days to analyze moisture-induced antigen instability. Results are shown in Table 5. Overall, lysine and sorbitol were excellent inhibitors of aggregation and antigenicity losses during exposure to moisture in the solid state and heat in solution. Trehalose and histidine can completely inhibit tetanus toxoid aggregation but some loss of antigenicity occurred.

TABLE 4
Effect of agents on fluorescent properties and antigenicity after
exposure to heat (45° C. and 22 days), relative to fresh toxin.

| | Fluorescent Properties | | Antigenicity |
|---|---|---|---|
| Agents | λₘₐₓ | I_{350/}I₃₂₉ | (%) |
| TT standard | 329 | 0.75 | 100 |
| None | 339 | 0.99 | 28 ± 1 |
| Histidine | 329 | 0.76 | 96 ± 22 |
| Lysine | 329 | 0.78 | 82 ± 12 |
| Trehalose | 331 | 0.82 | 44 ± 10 |
| Sorbitol | 330 | 0.78 | 83 ± 6 |
| Heparin | 332 | 0.83 | 61 |

TABLE 5
Effect of agents on fluorescent properties and antigenicity after exposure
to moisture (37° C. and 80% RH).

| | | | Soluble | | | | |
|---|---|---|---|---|---|---|---|
| | | | protein | Fluorescence | | | |
| | Soluble protein (%) | | (%) | properties | | Antigenicity | |
| | Stabilizing agent:TT | | Stabilizing | Stabilizing | | (%)/ | |
| | (1:1) | | agent:TT | agent:TT | | (Stabilizing | |
| Stabilizing | (15 days) | | (10:1) | (10:1) | | agent:TT | |
| agents | Commassie | A280 | (30 days) | λₘₐₓ | A₃₅₀/A₃₂₉ | ratio) | |
| TT standard | 100 | | | 329 | 0.75 | 100 | |
| Lyophilized | 100 | | | 327 | 0.73 | 100 | |
| TT standard | | | | | | | |
| None | 15 ± 1 | — | — | — | — | — | |
| Histidine | 104 ± 2 | — | 101 ± 5 | 329 | 0.72 | 38 ± 1 | (1:1) |
| | | | | | | 24 | (10:1) |
| Lysine | — | | 103 ± 1 | 329 | 0.76 | 81 ± 12 | (10:1) |
| Trehalose | 105 ± 5 | 106 ± 5 | 92 ± 4 | 328 | 0.73 | 69 ± 8 | (1:1) |
| | | | | | | 47 | (10:1) |
| Sorbitol | 97 ± 4 | 100 ± 6 | 87 ± 1 | 329 | 0.75 | 90 ± 8 | (10:1) |
| Heparin | 100 ± 1 | 106 ± 1 | 42 ± 5 | — | — | — | |

In another embodiment, trehalose is preferred for its ability to stabilize protein antigens during both lyophilization and microencapsulation processes. The possible mechanisms include stabilizing dried protein structure by replacing H-bonding sites on the protein previously occupied by water, increasing the glass transition temperature of the formulation, and promoting preferential hydration of the antigen. Additional experiments co-incorporated trehalose with histidine in the f-BSA-containing microspheres.

Before microsphere preparation, f-BSA was colyophilized with histidine only (the weight ratio of f-BSA to histidine was 5:1) or with both histidine and trehalose (the f-BSA/histidine/trehalose weight ratio was 5:0.5:0.5). Microspheres with 8% f-BSA loading were prepared by the o/o method. Without any stabilizers, f-BSA lost 70% of its solubility after 6 days of incubation, whereas f-BSA co-encapsulated with histidine and trehalose still remained 81 and 102% soluble after 28 days of incubation, respectively. Solubility data showing the effect of histidine and trehalose on f-BSA release is shown in Table 6.

TABLE 6
Effect of Histidine and Trehalose on f-BSA aggregation in
microspheres after exposure to 80% RH and 37° C.

| | Incubation | Soluble |
|---|---|---|
| Antigen Formulations | Condition | f-BSA (%)^{d} |
| Effect of histidine and trehalose | | |
| f-BSA o/o microspheres^{a} | 80% RH, 37° C., 6 days | 30 ± 5 |
| f-BSA + His o/o microspheres^{a,b} | 80% RH, 37° C., 6 days | 106 ± 10 |
| | 80% RH, 37° C., 28 days | 81 ± 5 |
| f-BSA + His + Tre o/o | 80% RH, 37° C., 6 days | 127 ± 7 |
| microspheres^{a,} | 80% RH, 37° C., 28 days | 102 ± 14 |

^{a}Theoretical protein loading was 8%; PLGA50/50 (iv = 0.64 dL/g) was used for microsphere preparation.
^{b}Soluble in PBST; data represent average ± SD, n = 3.

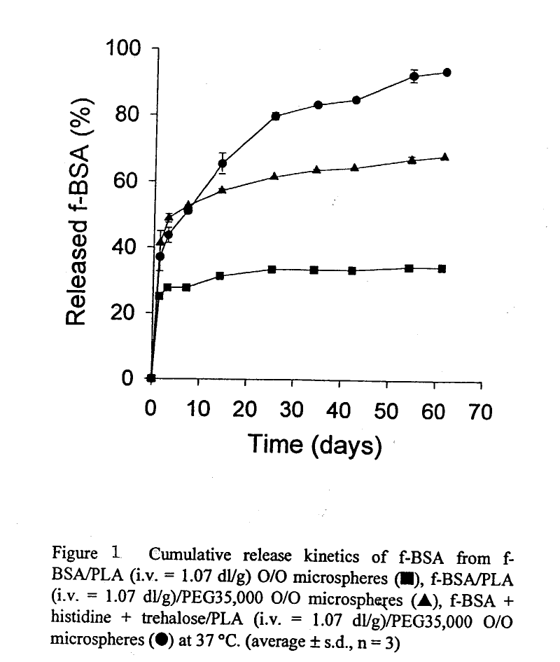

The effect of PEG blending and co-encapsulation of histidine and trehalose on the release kinetics of f-BSA is shown in FIG. 1. In the absence of PEG, histidine and trehalose, 34.5% of f-BSA was released out of the microspheres after 60 days of incubation in PBST at 37° C. The residual f-BSA remaining in microspheres formed insoluble aggregates. Blending PLA with PEG increased both the release rate and the total releasable amount of f-BSA. Addition of PEG (molecular weight 35,000 concentration (w/v) 6%) to the polymer increased the release of f-BSA to 68.1% after 60 days of incubation and the formaldehyde-mediated aggregates formed in microspheres were reduced to <15%. With co-encapsulation of histidine and trehalose (the ratio of f-BSA:histidine:trehalose was 1:0.5:0.5), the FMAP was completely halted. 93.5% of f-BSA was continuously released from the blended polymer and the unreleased f-BSA fraction remained soluble in microspheres.

### EXAMPLES

The following examples are for purposes of illustration only and are not intended to limit the scope of the claims which are appended hereto.

### Example 1

### Stabilization of Tetanus Toxoid Encapsulated in PLGA Microspheres

The purpose of this study is to mechanistically investigate the aggregation of tetanus toxoid (TT) in PLGA microspheres. Based on aggregation mechanisms and other inactivation sources, strategies were developed to improve the stability of encapsulated tetanus toxoid.

### Materials and Methods

### Chemicals and Materials

Poly(D, L-lactide-co-glycolide) 85/15 with inherent viscosity of 0.86 dl/g (Mw 146,000) and poly(D,L-lactide) with inherent viscosity of 1.07 dl/g (Mw 195,000) in CHCl₃ were from BPI (Birmingham, Ala.). TT (specific activity of 3300 Lf mg⁻¹ protein), was supplied by Chiron Co. (San Francisco, Calif.). TT010 monoclonal antibody and anti-TT guinea pig IgG were provided by National Institute of Biological Standard Control (UK). Goat anti-guinea pig IgG peroxidase conjugate and 2,2′-azino-di-3-ethylbenzthiazoline-6-sulphonate (ABTS) tablets were obtained from Sigma (St. Louis, Mo.). Coomassie Plus Protein reagent was obtained from Pierce (Rockford, Ill.). All other substances used were of pharmaceutical or analytical grade and purchased from commercial suppliers.

### Microsphere Preparation Method

Protein antigens were encapsulated by oil-in-oil emulsion and solvent extraction (o/o) method. In the o/o method, 300 mg of PLGA 85:15 polymer was dissolved in 1.2 ml acetonitrile (ACN). When MgCO₃ was co-encapsulated in the formulation, it was suspended in the polymer organic solution. 150 μl of concentrated protein aqueous solution was added in the acetonitrile-polymer solution, with gentle vortex, fine protein particles were precipitated out. This protein-polymer suspension was drop-wise added into cottonseed oil containing 1.6% (W/V) Span™ 85 stirred at 750 rpm with an overhead stirrer. After 5 hr, petroleum ether (b.p. 50-110° C.) was poured into the cottonseed oil bath to extract the acetonitrile from the polymer.

After an additional 15 min of stirring, the microspheres were filtered through 120 μm, washed with 250 ml of petroleum ether and lyophilized.

### The Effect of High Shear Stress, Organic Solvent and Aqueous-Organic Interface on Antigen Stability

To investigate the effect of organic solvent and high shear stress on the stability of TT during encapsulation, protein powder was suspended in acetonitrile and homogenized at 15,000 rpm for 1 min in an ice bath. Then the organic solvent was evaporated and protein residue was reconstituted in 1 mM pH 7.3 phosphate buffer for protein content and antigenicity assay. To test the potential inactivation of TT caused by the organic solvent precipitation during encapsulation, 1.5 ml of acetonitrile was added to 0.1 ml of 7 mg/ml of TT aqueous solution. After vortexing for 1 min, organic solvent and water was evaporated by a Vacufuge™ concentrator 5301 (Brinkmann Instruments, Inc., Westbury, N.Y.). Protein residues were reconstituted in 1 mM pH 7.3 phosphate buffer and assayed for soluble protein content and antigenicity.

### Determination of Antigen Loading

The amount of antigen encapsulated in microspheres was determined by two methods. The first method was to extract the protein from the microspheres by removing the polymer and determining the protein content by Coomassie plus assay. The second method determined encapsulated protein content by amino acid analysis after acid hydrolysis of microspheres. In the first method, acetone was added in microspheres to dissolve the polymer. The mixture was vortexed, centrifuged and then the supernatant was removed. After removal of polymer was repeated three times, the remaining protein pellet was reconstituted in phosphate buffer saline containing 0.02% Tween 80® (PBST) and protein content was determined by the Coomassie Plus method (Pierce Chem Co., Ill.) and ELISA. If any insoluble aggregates were observed in the reconstituted solution, the aggregates were collected, washed with distilled water and then freeze dried. These insoluble aggregates were reconstituted in denaturing agent (6 M Guanidine-HCl (GnCl)). Determination of any aggregates soluble in denaturing agent gave the amount of non-covalently bonded aggregates. With the further addition of reducing agent (10 mM DTT+1 mM EDTA), any disulfide-bonded aggregates were dissolved. The total dissolved portion in denaturing and reducing agents gave the total amount of non-covalent and disulfide-bonded aggregates.

In the second method, 2-3 mg microspheres or protein aggregates were weighed and dissolved in 6 N HCl/TFA (1:1), then an aliquot of the solution was hydrolyzed under 6 N HC1 vapor at 125° C. for 24 hrs. The Applied Biosystems (ABI) Model 420 H was used to derivatize hydrolyzed amino acids with phenylisothiocyanate (PITC) at alkaline pH to form PTC-amino acid derivatives. The PTC-amino acid derivatives were automatically injected onto an ABI model 130A HPLC and analyzed.

### Evaluation of Model Antigen Release from Microspheres

15 mg of microspheres were incubated in 0.5 ml PBST or PBST containing 0.2% BSA (PBSTB) at 37° C. Release media were collected periodically and the soluble protein content was determined by Coomassie plus assay or ELISA. At the end of release, microspheres were collected and remaining soluble protein or any insoluble protein aggregates in the microspheres was analyzed as described in the section Determination of antigen loading.

### ELISA for Tetanus Toxoid

A sandwich ELISA was used to determine the antigenic content of tetanus toxoid using a monoclonal antibody as the capture antibody. Wells of microtitre plates were coated with 100 μl of 1 μg ml⁻¹ of the capture antibody in coating buffer overnight at 4° C. After blocking, and washing three times with phosphate buffer saline containing 0.05% Tween 20 (PBST), serial dilutions of reference tetanus toxoid and samples in PBSTB were added and incubated for 2 hr at 37° C. After washing three times with PBST, plates were then incubated with purified anti TT guinea pig immunoglobulin (IgG) for 2 hr at 37° C., followed by incubation with the goat anti-guinea pig IgG-horseradish peroxidase (HRP) conjugate in PBSTB for 1 hr at 37° C. Finally, the substrate solution containing 0.5 mg ml⁻¹ of ABTS and 0.04% H₂O₂ in 0.05 M citric acid, pH 4.0 was added. Color was allowed to develop for 25-30 min at room temperature and absorbance was read at 405 nm.

### Results and Discussion

When concentrated TT aqueous solution was precipitated by ACN, no solubility loss of TT was observed whereas there was a slight decrease in antigenicity (87% retained). These results suggested that the O/o encapsulation process would denature TT, but within an acceptable limit. However, other encapsulation techniques may offer lesser denaturation. The encapsulation has not been optimized

TABLE 7
Characteristics of microsphere formulations.

| | | | | Aggregates/loading (%) | |
|---|---|---|---|---|---|
| | | | | | Non- |
| | | | | | covalent |
| | | | Amino | | and |
| | Proten | Loading (%) | acid | Non- | disulfide- |
| Stabilizer | content | Antigenicity | analysis | covalent | bonded |
| none | 1.5 ± 0.1 | 0.82 ± 0.002 | 1.63 | 0.12 ± 0.02 | 0.3 ± 0.1 |
| TT:lysine (1:3), | 1.7 ± 0.4 | 1.4 ± 0.1 | 1.72 | 0.04 ± 0.00 | 0.07 ± 0.03 |
| 3% MgCO₃ | | | | | |
| TT:lysine:sorbitol | 2.1 ± 0.2 | 1.1 ± 0.2 | 2.28 | 0.08 ± 0.00 | 0.13 ± 0.04 |
| (1:1.5:1.5), | | | | | |
| 3% MgCO₃ | | | | | |
| TT:lysine:trehalose | 2.5 ± 0.1 | 1.5 ± 0.4 | 2.8 | 0.06 ± 0 | 0.08 ± 0.02 |
| (1:1.5:1.5), | | | | | |
| 3% MgCO₃ | | | | | |

TABLE 7 shows the characteristics of microsphere formulations. Theoretical TT loading in PLGA microspheres was roughly 3%. TT loading as determined by the Coomassie plus assay was 1.5%, indicating that 50% encapsulation efficiency was obtained by the O/o encapsulation method herein described. Co-encapsulation of stabilizing agents increased the encapsulation efficiency of TT. As shown in Table 7, when lysine and MgCO₃ were co-encapsulated, protein content loading was 1.7% and 80% of encapsulated TT retained antigenicity. When sugars were co-encapsulated with lysine and MgCO₃ in PLGA microspheres, a substantial increase of TT loading was noted. In addition, the amount of insoluble aggregates was significantly reduced with the co-encapsulation of stabilizing agents, indicative of their stabilization effect during encapsulation.

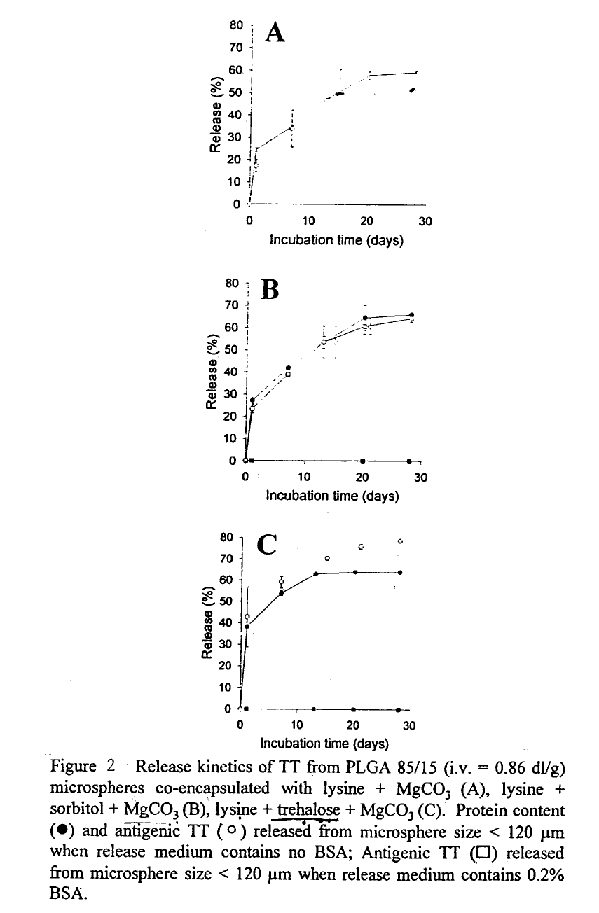

FIGS. 2a, 2b, and 2c show release kinetics of formulations containing different stabilizing agents. Compared to TT microspheres without additives, a promising release pattern, as determined by either Coomassie plus assay or ELISA, was exhibited with all three formulations containing additives (TT/lysine/MgCO₃, TT/lysine/sorbitol/MgCO₃, TT/lysine/trehalose/MgCO₃). The release pattern consisted of an initial burst followed by a constant release of TT over 28 days. For the formulation containing trehalose, a relatively higher initial burst was observed.

TABLE 8
Analysis of residual TT in PLGA microspheres after
28-day incubation.

| | | | Non- | Non-covalent | |
|---|---|---|---|---|---|
| | Release | | covalent | and disulfide- | |
| | of | Soluble | aggregates | bonded | Recovery |
| Stabilizers | TT (%) | TT (%) | (%) | aggregates % | (%) |
| none | 12 ± 2 | 2 ± 1 | 10 ± 3 | 19 ± 6 | 33 ± 6 |
| Lysine + | 60 ± 1 | 1 ± 1 | 25 ± 3 | 40 ± 1 | 100 ± 3 |
| MgCO₃ | | | | | |
| Lysine + | 66 ± 1 | 1 ± 1 | 10 ± 2 | 20 ± 1 | 87 ± 2 |
| Sorbitol, | | | | | |
| MgCO₃ | | | | | |
| Lysine + | 64 ± 2 | 2 ± 1 | 11 ± 1 | 18 ± 3 | 84 ± 3 |
| Trehalose, | | | | | |
| MgCO₃ | | | | | |

Table 8 shows the analysis of residual TT protein remaining in PLGA microspheres after 28 days incubation. With co-encapsulation of lysine/MgCO₃, lysine/sorbitol/MgCO₃, and lysine/trehalose/MgCO₃, total released amount of TT increased to above 60%. The analysis of the protein residue remaining after the 4-week incubation showed that in the presence of lysine, lysine/sorbitol and lysine/trehalose, the amount of formaldehyde-mediated aggregates of TT was significantly reduced or even absent (for the formulation containing lysine but no sugar).

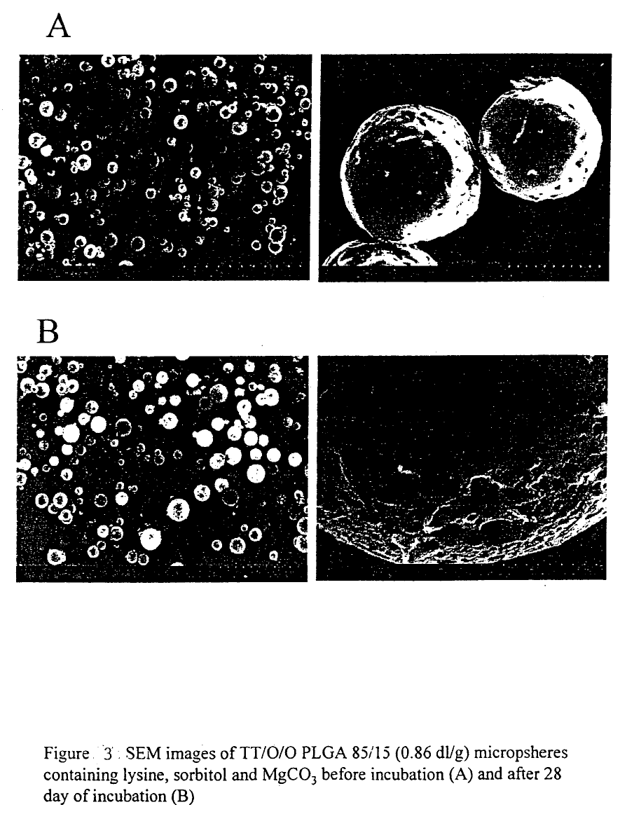

The morphology of microspheres co-encapsulated with lysine and sorbitol as shown by SEM images in FIGS. 3a and 3b. Co-encapsulation of stabilizing agents does not change the microsphere morphology in comparison to microspheres formed without stabilizing stabilizing agents.

### Example 2

### Effect of Stabilizing Agents on the Structure, Antigenicity and Aggregation of Tetanus Toxoid

The purpose of this study is to identify efficient stabilizing agents that not only inhibit tetanus toxoid aggregation but also afford retention of its antigenicity under simulated deleterious conditions, i.e., elevated temperature and humidity.

### Chemicals and Materials

Tetanus toxoid (TT) (specific activity of 3300 Lf mg⁻¹ protein), was supplied by Chiron Co. For the ELISA, TT010 monoclonal antibody and purified guinea pig anti-IT IgG were provided by National Institute of Biological Standard Control (NIBSC, UK). Rabbit anti-guinea pig IgG peroxidase conjugate and 1,2′-azino-di-3-ethylbenzthiazoline-6-sulphonate (ABTS) tables were obtained from Sigma Chemical Co. (St. Louis, Mo.). Coomassie Plus Protein reagent was obtained from Pierce Chemical Co.(Rockford, Ill.). Tested stabilizing agents were trehalose, sorbitol, heparin, lysine, histidine.

### Moisture-Induced Antigen Instability

Tetanus toxoid solution was diluted to ˜1 mg/ml and dialyzed (10,000 molecular weight cut-off membranes) against 1 mM phosphate buffer at pH 7.3 (4° C.). Stabilizing agents, at a 1 or 10-fold weight excess, were added to protein solutions just before lyophilization. Protein samples were flash frozen in liquid N₂ and lyophilized for 48 hr. Lyophilized antigen samples (0.25 mg) were incubated at 37° C. and 80% relative humidity (R.H.). After 15 or 32 days, incubated samples were reconstituted in 1 mM phosphate buffer. The undissolved antigen was removed by centrifugation and the soluble antigen concentration was determined by Coomassie Plus Protein assay or its absorbance at 280 nm. Protein samples, in which no significant amount of insoluble aggregates formed, were further analyzed for structural integrity and antigenicity.

### Heat-Induced Antigen Instability

Stabilizing agents were added to 0.67 mg/ml protein solutions at a 10-fold weight excess and sealed in ampules. To test the effect of basic additives, TT (1 mg/ml), and a solution of lactic (22 mM) and glycolic acids (24 mM) were combined and the pH was adjusted to between 2.9 and 8 with MgCO₃. Protein solutions in ampules were incubated at 45° C. for 22 days and then analyzed as described above.

### Structural Analysis of TT

Structural changes in antigen samples were determined by measuring circular dichroic (CD) and fluorescence spectra. Far-UV CD spectra were taken with a J-810 Jasco spectropolarimeter (Hachioji, Japan) at room temperature. Samples were placed in a quartz cell with a 0.1 cm pathlength for measurement of far ultraviolet (200-250 nm) spectra at 1 nm intervals, which reflects the secondary structure change in TT. The spectra were corrected for buffer control without antigen samples. Tertiary structural changes in TT were monitored by fluorimetry (Fluoromax, N.J.) using different excitation and emission wavelengths. Toxoid solutions were excited (λₑₓ) at 280 nm and the ratio of emission intensity at 350 nm over that at 329 nm (I₃₅₀/I₃₂₉) was studied [Johansen et al. 1998]. Both changes in emission wavelength maxium (λ_{max) and I}₃₅₀/I₃₂₉ are sensitive indices of conformational changes.

### ELISA of TT

A sandwich ELISA was used to determine the antigenic content of tetanus toxoid using monoclonal antibody as the capture antibody. Wells of microtitre plates were coated with 100 μl of 1 μg ml⁻¹ of the capture antibody in coating buffer overnight at 4° C. After blocking and washing three times with phosphate buffer saline containing 0.05% Tween 20 (PBST), serial dilutions of reference TT and samples in phosphate buffer saline containing 0.05% Tween 20 and 1.5% bovine serum albumin (PBSTB) were added and incubated for 2 hr at 37° C. After washing three times with PBST, plates were then incubated with purified anti TT guinea pig immunoglobulin (IgG) for 2 hr at 37° C., followed by incubation with the goat anti-guinea pig IgG-horseradish peroxidase (HRP) conjugate in PBSTB for 1 hr at 37° C. Finally, the substrate solution containing 0.5 mg ml⁻¹ of ABTS and 0.04% H₂O₂ in 0.05 M citric acid (pH 4.0) was added. Color was allowed to develop for 30 min at room temperature and absorbance was read at 405 nm.

Tables 4 and 5 show the results of stabilizing agents on the antigenicity, structure and aggregation of TT . With no stabilizing agents added, after incubation at 45° C. for 22 days, the antigenicity of TT was decreased to 28%. With the addition of all tested stabilizing agents, TT antigenicity was significantly improved. For example, TT with histidine, lysine and sorbitol retained above 80% of antigenicity after 22 days of incubation. Table 5 shows that after 32 days incubation, TT incubated with histidine and lysine still remained 100% soluble after incubation.

### Example 3

### Comparison of Encapsulation Methods

### Chemicals and Materials

Poly (D,L-lactide-co-glycolide) (PLGA) 50/50 with inherent viscosity (iv) of 0.20 and 0.64 dL/g in hexafluoroisopropanol and poly(D,L-lactide)(PLA) with iv of 1.07 dL/g in CHCl₃ were from BPI (Birmingham, Ala.). Bovine serum albumin (BSA;A-3059, Lot 32H0463) was purchased from Sigma Chemical Company (St. Louis, Mo.). Formalinized bovine serum albumin was developed by treating BSA with formaldehyde for 3 weeks. All other biochemicals and chemicals were of analytical grade or purer and obtained from commercial suppliers.

### Preparation of Polymeric Microspheres

The polymeric microspheres were prepared by either a water-in-oil-in-water double emulsion-solvent evaporation (w/o/w) method or an oil-in-oil single emulsion-solvent extraction (o/o) method. In the w/o/w method, an aqueous solution of antigen in 1 mM pH 7.3 phosphate buffer was emulsified into a methylene chloride-PLGA 50/50 solution by homogenization. Then 1% poly(vinyl alcohol) (PVA) aqueous solution was added to the first w/o emulsion and the second w/o/w emulsion was formed by vortex at high speed. This emulsion was poured into a hardening aqueous bath containing 0.3% PVA and stirred for 3 h at room temperature and atmospheric pressure. The hardened microspheres were collected by centrifugation, washed with double distilled water, and lyophilized over 2 days.

In the anhydrous o/o method, dried protein powders were directly suspended in acetonitrile-polymer solution. The lyophilization protocol to prepare the dried protein powder was as follows: protein aqueous solutions in 1 mM pH 7.3 phosphate buffer were flash frozen in liquid N₂ and placed on a Labconco Freeze Dryer (Kansas City, Mo.) operating at 30 μM of Hg pressure and a condenser temperature of −46° C. Typically, protein samples were removed after 48 hr. Then, a uniform suspension of sieved antigen powder (<45 μm) in acetonitrile-polymer solution was homogenized at 15,000 rpm in an ice bath. The antigen suspension was added in a dropwise manner into the continuous phase [cottonseed oil containing 1.6% (w/v) Span™ 85] stirred at 750 rpm with an overhead stirrer. After 5 h, petroleum ether (bp, 50-110° C.) was poured into the cottonseed oil bath to extract the acetonitrile from the polymer. After additional 15 min. of stirring, the microspheres were filtered, washed with 250 mL of petroleum ether and lyophylized.

The comparison for enhanced stability afforded by the method of encapsulation was measured by the stability of f-BSA in PLGA. To achieve higher loading, PLGA 50/50 (inherent viscosity=0.64 dL/g) was selected to encapsulate model antigens and the theoretical loading of f-BSA was 8% in both o/o and w/o/w encapsulation methods. In the o/o method, the polymer concentration in acetonitrile was 10%, and 98±4% (n=3) encapsulation efficiency was achieved. In the w/o/w method, polymer concentration in methylene chloride was 30% and encapsulation efficiency was only 49±3% (n=3). The influence of encapsulation methods on f-BSA stability at 97%relative humidity and 37° C. was also measured. In the w/o/w formulation, after 16 days of incubation, only 50±2% of f-BSA remained soluble in microspheres, whereas 112±1% of f-BSA remained soluble in microspheres. See Table 3.

### Example 4

### Inhibition of the Formaldehyde-Mediated Aggregation Pathway (FMAP) Using Stabilizing Agents

The inhibition of the FMAP was tested by colyophilizing a series of nucleophiles (amino acid) with BSA and f-BSA before examining protein aggregation in the bovine serum solubility assay. Before lyophilization, solution pH was confirmed to be neutral (pH 7.3±0.1). The nucleophiles examined included L-glycine (Gly), L-alanine (Ala), L-cysteine (Cys), L-histidine (His), L-glutamine (Gln), L-glutamic acid (Glu), L-lysine (Lys), and L-arginine (Arg). Lyophilized protein samples were incubated with 50% of water (relative to protein weight) at 37° C. After 4 days of incubation, the effect of amino acids on the antigen aggregation extent was determined as seen in Table 9, in the absence of any amino acid, only ˜10% f-BSA remained soluble in aqueous reconstitution media. However, all the amino acids colyophilized with f-BSA inhibited the aggregation of f-BSA to some extent. The greatest inhibition of f-BSA aggregation was recorded in the presence of Lys and His. Both amino acids completely prevented f-BSA aggregation after 4 days. By 35 days of incubation, f-BSA with Lys lost ˜65% of its solubility, whereas f-BSA with His was still completely soluble after reconstitution. The ability to block F-BSA aggregation was grouped roughly in the following order: His>Lys>Gly>Arg>Gln>Cys>Ala. Soluble antigen was analyzed either by using the Coomassie Plus method (Pierce Chemical Co.) or by absorbance at 280 nm.

TABLE 9
Effect of amino acids on the aggregation of BSA and f-BSA.

| | Soluble protein % (average ± SD, n = 3 | |
|---|---|---|
| | BSA | f-BSA |
| | (after 35-day | (after 4-day |
| Amino acid | incubation) | incubation) |
| No amino acid (control) | 100 ± 0 | 11 ± 2 |
| With amino acid^{a} | | |
| Nonpolar chains | | |
| Glycine | 100 ± 0 | 95 ± 2 |
| _{L}-Alanine | 100 ± 0 | 26 ± 5 |
| Uncharged polar side chains | | |
| _{L}-Glutamine | 100 ± 0 | 78 ± 2 |
| _{L}-Cysteine | 6 ± 2 | 65 ± 13 |
| _{L}-Histidine | 100 ± 0 | 100 ± 0^{b} |
| Charged polar side chains | | |
| _{L}-Glutamic acid | 100 ± 0 | 69 ± 8 |
| _{L}-Arginine | 100 ± 0 | 86 ± 4 |
| _{L}-Lysine | 100 ± 0 | 100 ± 0^{c} |

^{a}The weight ratio of protein to amino acid was 5:1.
^{b}After 35-day incubation, 100 ± 0% of f-BSA remained soluble.
^{c}After 35-day incubation, 35 ± 0% of f-BSA remained soluble.

### Example 5

### Stabilization and Controlled Release of Bovine Serum Albumin Encapsulated in Poly(D,L-Lactide) and Poly(ethylene glycol) Microsphere Blends.

This example is taken from U.S. application Ser. No. 09/738,961 herein incorporated by reference.

### Materials And Methods

### Chemicals

Poly(D,L-lactide) with inherent viscosity of 1.07 dug in CHCl₃was from BPI (Birmingham, Ala.). Poly(ethylene glycol) with molecular weights of 10,000 and 35,000 were obtained from Aldrich Chem, Co. (Milwaukee, Wis.) and Fluka, respectively, Bovine serum albumin: (A-3059, Lot 32H0463) was purchased from Sigma Chemical Co. (St. Louis, Mo.). Protein molecular weight and pI standards for electrophoresis were from Pharmacia LKB (Piscataway, N.J.). All other biochemicals and chemicals were of analytical grade or purer and obtained from commercial suppliers.

### Microsphere Preparation

The polymeric microspheres were prepared by an anhydrous o/o method. First, PLA and PEG at various weight ratios were co-dissolved in acetonitrile at a total polymer concentration of 20% (w/v). Sieved BSA (<20 μm) was suspended in acetonitrile-polymer solution and homogenized at 15,000 rpm in an ice bath. Then the antigen suspension was added drop-wise into the continuous phase (cottonseed oil containing 1.6% (W/V) Span™ 85) stirred at 750 rpm with an overhead stirrer. After 5 hr, petroleum ether (b.p. 50-110° C.) was poured into the cottonseed oil bath to extract the remaining acetonitrile from the polymer. After an additional 15 min of stirring, the microspheres were filtered, washed with 250 ml of petroleum ether and lyophilized.

### Microsphere Characterization

### Morphology and Particle Size Determination

The microspheres were coated with gold-palladium by using PELCO MODEL 3 SPUTTER COATER 91000. Surface morphology of the microspheres was examined by a Philips XL Scanning Electron Microscope. Particle size was estimated by averaging diameters of 50 microspheres.

### Polymer Composition Analysis by IR

The composition of microspheres prepared from different blends of PLA and PEG was analyzed by infrared spectroscopy. A Nicolet protege 460 was used to obtain the spectra (32 scans per sample, over 600-4000 cm⁻¹ ) for the samples. A series of PLA and PEG physical mixtures with different weight ratios was used to make a calibration curve. Samples were dissolved in chloroform and casted into a sodium chloride cell. The composition of the microparticles was estimated by comparing peak height ratios corresponding to the carbonyl (C═O) band of PLA at 1757 cm⁻¹ and the CH₂ band at 2876 cm⁻¹ due to the PEG component, and assuming a negligible content of Span™ 85 surfactant in microspheres.

### Polymer Phase Behavior Analysis by DSC

Samples (3-5 mg) were loaded into aluminum pans and DSC thermograms were recorded by a Perkin-Elmer DSC 7 Differential Scanning Calorimeter. Nitrogen gas was the sweeping gas and the heating rate was 20° C./min.

### Determination of Microsphere Loading

The amount of antigen encapsulated in microspheres was determined by recovering the protein from the microspheres. First, acetone was added to the microspheres to dissolve the polymer. The mixture was vortexed, centrifuged and then supernatant was removed. After the removal of polymer was repeated three times, the remaining protein pellet was air dried and reconstituted in phosphate buffer saline pH 7.4 containing 0.02% Tween 80® (PBST) and protein content was determined by the Coomassie Plus method (Pierce Chem Co., IL).

### Evaluation of Model Antigen Release from Microspheres

Samples of 10 mg microspheres were suspended in 1 ml PBST. The suspension was incubated at 37° C. under mild agitation. At pre-selected intervals, release media were removed for determination and replaced with fresh buffer. The amount of protein released was assayed by the Coomassie Plus method (Pierce Chem. Co., IL). At the end of release, microspheres were collected and remaining soluble protein in the microspheres was analyzed as described in the section Determination of antigen loading.

Any insoluble protein aggregates were collected by centrifugation and reconstituted in denaturing a gent (8 M Urea or 6 M Guanidine-HCI (GnCl)). Determination of any aggregates soluble in denaturing agent gave the amount of noncovalently bonded aggregates. With the further addition of reducing agent (10 mM DTT+1 mM EDTA), any disulfide-bonded aggregates were dissolved. The total dissolved portion in denaturing and reducing agents gave the total amount of non-covalent and disulfide-bonded aggregates.

### pH Change in the Release Medium During Release

The pH of the release medium was monitored by a Coming 430 pH meter (Coming Inc., NY) at each sampling interval.

### Water Uptake of Microspheres

After incubation at 97% relative humidity and 37° C., samples were taken out and weighed immediately. The water uptake of microspheres was estimated by:

Water uptake (%)=(W₁-W₂)/W₂×100%

Where W₁ and W₂ are the weights of the hydrated microspheres and microspheres before incubation, respectively. No corrections were made for inter-particle water content in W₁ or the water content within lyophilized microspheres in W₂.

### Structural Analysis of Encapsulated BSA

At the end of release period, the integrity of remaining BSA in the polymer was determined by both sodium dodecyl sulfate polyacrylamide gel electrophoresis (SDS-PAGE) and isoelectric focusing (IEF) analysis, which were performed on a Pharmacia PhastSystern (Pharmacia Biotech.) according to the file no. 110 and 100 in the Phastsystem™ User Manual, respectively. In both analyses, Coomassie staining file no. 200 was used. The secondary structure of antigen samples was determined lay measuring circular dichroic (CD) spectra. The spectra were taken with a J-500A Jasco spectropolarimeter (Hachioji, Japan) at room temperature. The tertiary structure of protein samples was analyzed by measuring the intrinsic fluorescence emission spectra. Fluorescence emission spectra (300-500 nm for BSA) were obtained on a Perkin-Elmer LS5OB luminescence spectrometer scanned at 240 nm/min. The excitation wavelength for BSA was set to 295 nm. Details of these procedures were as described previously (Jiang and Schewendeman, Biotech. Bioeng. 70(5): 507-517, 2000).

### Results

### Microsphere Composition and Phase Behavior Analysis

By estimating the PEG content in the blend with a calibration curve generated from PLA and PEG physical mixtures with different weight ratios, complete incorporation of PEG in PLA matrix by the o/o encapsulation method was indicated (data not shown).

### Microsphere Morphology

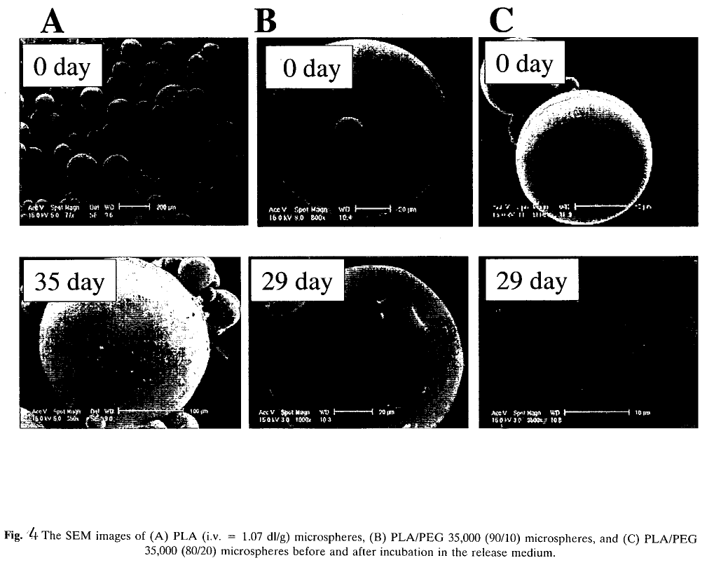

As seen in FIGS. 4a, 4b, and 4c, after preparation, microspheres with different weight ratios of PLA and PEG had spherical and smooth surfaces. An average size of ˜100 μm was recorded for these microsphere preparations. After 35 days of incubation, microspheres prepared from 100% PLA remained intact with a smooth surface. With the blend of PEG, the microsphere structure still remained intact, but a small amount of pores appeared on the PLA/PEG microspheres surface. With higher PEG blend, more pores became visible. In addition, the microsphere surface showed indentations, which may have occurred during drying of the particles before analysis. The SEM images suggested that the incorporation of PEG into PLA created more channels in the microspheres, which may have increased the permeability to the encapsulated protein. In addition, the microsphere surface likely consisted of a PLA-rich phase, whereas the interior of microspheres was likely PEG-rich. Otherwise, more pores created by PEG solubilization would be expected on the microsphere surface. The PLA-rich surface phenomenon is possibly due to the higher hydrophobicity and longer chain of PLA, which could have caused selective PLA precipitation at the surface during the O/o microsphere preparation.

### Release Kinetics and Stability of BSA in the PLA/PEG Microspheres

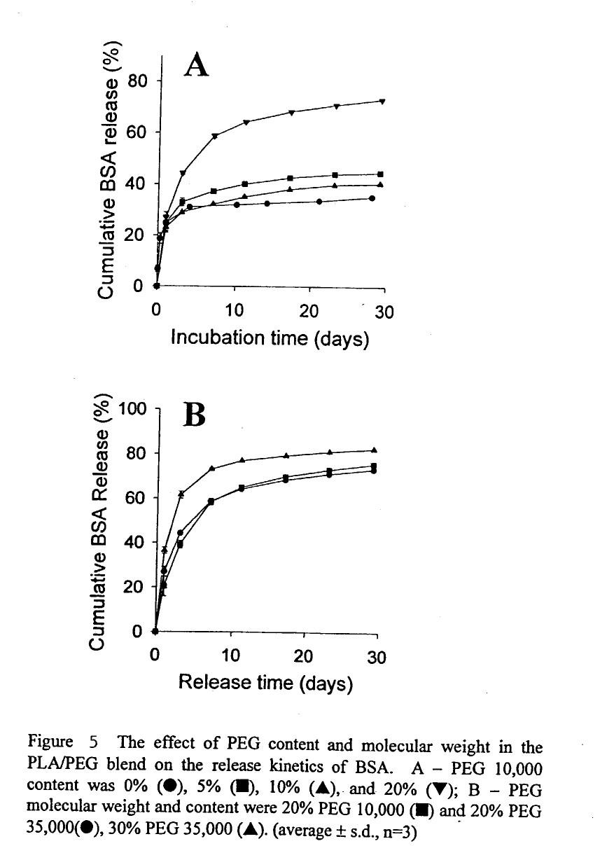

To investigate the effect of PEG in the PLA/PEG microspheres, microspheres with different weight ratios of PEG 10,000 to PLA were prepared and the BSA controlled release was monitored in PBST at 37° C. Formulations for PLA/PEG microspheres are detailed in Table 2 and are herein described by the Form Code listed in Table 2. Theoretical BSA loading of all these formulations was 5% and encapsulation efficiency vas invariably between 90% and 100%. As seen in FIG. 5a, when PEG content was less than 10% of polymer weight, similar release kinetics of BSA from microspheres was observed and less than 45% of BSA was released after a 4-week incubation. When PEG content was raised to 20%, the total releasable amount of protein was significantly increased to 60%. In addition, the effect of PEG molecular weight on protein release was also evaluated. As seen in FIG. 5b, BSA had almost identical release kinetics in microspheres irrespective of whether PEG 10,000 and PEG 35,000 was used (the weight ratio of PEG/PLA was 20:80). When PEG 35,000 content was increased to 30% in PLA/PEG microspheres, a higher burst release of BSA was observed.

The residual BSA remaining in these devices after the 4-week release interval (45 days for formulation without PEG, i.e., formulation o) was analyzed. For the formulation o, of the original encapsulated protein, 15% was still water-soluble and 25% of BSA had become water-insoluble aggregates in the residue. Most of the aggregates were soluble in a denaturing solvent (6 M urea), indicating their non-covalent character. When 5% of PEG was incorporated in PLA (formulation a), soluble BSA remaining in microspheres was increased to 30%, and the non-covalent aggregates were 41% of the original encapsulated BSA. When PEG content was increased to 10% (formulation b), 36% of the protein formed insoluble aggregates. Besides non-covalent aggregates, a small fraction of disulfide-bonded aggregates (soluble in 10 mM DTT) was also formed. However, no insoluble BSA aggregates were observed in formulations containing more than 20% PEG.

The integrity of the soluble BSA recovered from the polymer (28-day incubation) was further examined by SDS-PAGE. Some peptide fragments were observed in formulations a and b, indicating mild peptide bond hydrolysis occurred during incubation with PEG content of 5 or 10%. In contrast, soluble BSA recovered from formulations containing more than 20% PEG showed a very similar band with standard BSA and no degradation product bands were noticeable. Soluble BSA recovered from formulations c, d and e was further examined by Isoelectric Focusing. No pI alterations in BSA were observed in these samples. Likewise, secondary and tertiary structure of BSA was similar to standard BSA control. Hence, the structure of BSA in formulations c, d, and e was retained within the polymer for one month.

### Mechanisms of BSA Stabilization in the PLA/PEG Microspheres

One-month continuous release of stable BSA from microspheres was achieved when PEG content in the PLA/PEG blends was above 20%. As identified previously, an acidic microclimate and intermediate moisture levels are the two major factors which cause non-covalent aggregation and peptide-hydrolysis of BSA in PLGA 50/50 microspheres. Without wishing to be bound to any particular theory, the blend of PEG with PLA appears to improve the microclimate, i.e., by avoiding the acidic microclimate and increasing the water content to stabilize BSA encapsulated in microspheres.

The pH change of the release medium when the PLA/PEG formulations were incubated at 37° C. and PBST (pH 7.4) were examined. Unlike PLGA 50/50, which showed a dramatic pH drop in the release medium after 4-week incubation (Zhu et al., Nat. Biotechnol. 18(1): 52-57, 2000; Zhu and Schwendeman, Pharm. Res. 17(3): 351-357, 2000), both PLA and PLA/PEG formulations remained at a relatively neutral pH (above 7) in the release medium over 29 days of incubation. However, a slightly lower pH in the release medium incubated with PLA/PEG formulation than that in PLA was observed (−0.1 −0.2 pH units difference). This result suggests that some acidic degradation products diffused out of the polymer through the water channels formed by PEG in PLA/PEG formulation. In addition, by using a previously reported method (pH determination of polymer solution in the mixture of ACN and water) (Shenderova et al., Pharm. Res. 16(2): 241-248, 1999), the pa_{H}* inside formulation d before and after 30-day incubation was determined as 6.5 and 5.4, respectively, suggesting a very small accumulation of acid in the polymer. In contrast, PLGA 50/50 microspheres were reported to reach pa_{H}* of 3 after similar incubation time (Shenderova et al., Proceed. Int'l. Symp. Control. Rel. Bioact. Mater. 27: S-0413.). These results demonstrate that acid build-up may be largely reduced in the PLA/PEG blend formulation.

The water content difference in formulations during release was compared by performing a water uptake kinetics study of microspheres at 97% R.H. Under controlled humidity, microspheres will adsorb water vapor and potential water uptake of different formulations during release can be compared. PEG 35,000 showed a strong water uptake. On the second day, the water content in PEG 5,000 blank microspheres was almost 120% of the dry microsphere weight. Upon blending PEG in the formulation, the water uptake rate was significantly increased. The higher the PEG content, the higher the increase in water uptake. Microspheres containing 20% PEG had almost twice the amount of water uptake; relative to those with 10% of PEG in the humid environment. When microspheres are incubated in the release medium, higher water content in the PLA/PEG blend is expected. The presence of 5% BSA did not increase water uptake rate significantly in the blend formulation. The water uptake in the blend was likely overwhelmed by the strong water sorption by PEG.

The above results demonstrate that a less acidic and more hydrophilic microenvironment is achieved in the PLA/PEG blend. Again without wishing to be bound by any particular theory, maintenance of a relatively neutral microclimate in PLA/PEG blend formulation can be attributed to several factors. First, few acidic species are produced during early incubation due to the slow degradation of PLA. The rate constant of PLA degradation at 37° C. in water has been reported to be roughly 0.012 day⁻¹, much slower than PLEA 50:50, 75:25, 85:15 with rate constants of 10.55, 0.103, and 0.026 day⁻¹, respectively (Magre and Sam, J. Control. Rel. 48: 318-319, 1997). In addition, prior to hydration, the polymer acid content was determined as 21 and 4.2 nmol/mg for the PLGA 50/50 used in our previous study and PLA used here, respectively (Shenderova, Ph.D Thesis. The Ohio State University, 2000). Therefore, the total amount of acidic species in PLA should be less than PLGA either during encapsulation or during hydration. Second, the blend of PEG with PLA significantly increases the water content in the formulation, which is expected to dilute the acidic species even further. Third, dissolution of PEG in the release medium may create more water channels, thereby increasing the diffusion for acidic species out of the polymer and for buffering species into the PLA matrix.

As a result, a less acidic microclimate is formed in the PLA/PEG blend. When PEG content is less than 10% in the blend formulations, non-covalent aggregates and peptide fragments of BSA were still observed. This is possibly due to regional acidity in the polymer which caused BSA degradation. Slowly produced polymer degradation products in certain regions cannot be diluted, or cannot diffuse out of the polymer because of insufficient water channels, resulting in regionally low pH. With increasing amount of PEG in the blend, a relatively neutral microclimate may be gradually attained. Although slight pH decreases within the polymer may be still detected in the blend containing 20% PEG, the amount is not significant enough to cause non-covalent aggregation and peptide-hydrolysis of BSA.

The stabilization of BSA in the PLA/PEG microspheres may also be attributed in part to the increased water content in the formulation. It was reported that the aggregation of BSA at acidic pH (pH=2) exhibits a pronounced bell-shape distribution with maximum aggregation corresponding to a water uptake of roughly 100 g water/100 g dry protein. When water uptake increased to 500%-1000%, aggregation of BSA declined sharply (Zhu, Ph.D thesis. The Ohio State University, 1999). In the blend formulation containing 20% of PEG, when incubated at 97% R. H. for 1 week, the water content in the microspheres is approximately 25%. Assuming all the water is available for BSA (BSA loading is 5%) in microspheres, the water uptake in the BSA phase is approximately 500%. During release, the water uptake in microspheres was expected to be higher than 500%. Thus, in addition to the minimal acid content, the aggregation of BSA may have been minimized by the high amount of imbibed water in the microenvironment.

Moreover, PEG may potentially interact with the hydrophobic groups of BSA and induce BSA unfolding. It was reported that PEG of low MW 1000 and 4000 interacts favorably with hydrophobic sides chains of human serum albumin (HSA), leading to a stabilization of the unfolded state (Farruggia, Int'l J. Bio. Macromol., 20:43-51, 1997). To test the interaction of high molecular weight PEG with BSA, the GnCl unfolding curve of BSA with the addition of PEG 10,000 and PEG 35,000 (weight ratio of BSA:PEG=1:5) was determined by fluorescence spectroscopy. Similar unfolding curves were observed in three preparations. The conformational stability of BSA was therefore likely not affected by the addition of PEG 10,000 and PEG 35,000 with 1:5 ratio of BSA to PEG. By using the PLA/PEG blend, a one-month continuous release of BSA was achieved with the absence of insoluble aggregates and peptide hydrolysis.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention. Molecular weights of polymers are weight average molecular weights unless indicated otherwise.

What is claimed is:

1. A delivery system for sustained release of formaldehyde-treated vaccine antigens in a biocompatible polymeric matrix, said system comprising:
a biocompatible polymer matrix;
at least one formaldehyde-treated vaccine antigen; and
at least one organic formaldehyde-interacting stabilizing agent in an amount effective to stabilize the at least one formaldehyde-treated vaccine antigen and to inhibit the formation of aggregates, wherein the at least one stabilizing agent and the at least one formaldehyde-treated vaccine antigen are encapsulated within the biocompatible polymer matrix.

2. The delivery system of claim 1 wherein the at least one stabilizing agent inhibits the formaldehyde-mediated aggregation of formaldehyde treated bovine serum albumin in the bovine serum albumin solubility assay by greater than 60%.

3. The delivery system of claim 1 wherein the at least one stabilizing agent is selected from the group of formaldehyde-interacting amino acids consisting of lysine, histidine, arginine, and glutamine.

4. The delivery system of claim 1 wherein the biocompatible polymer matrix is biodegradable.

5. The delivery system of claim 4 wherein the biodegradable polymer matrix is selected from homo-polymers and co-polymers derived from lactic and glycolic acids or lactides and glycolides.

6. The delivery system of claim 1 further comprising at least one basic stabilizing additive.

7. The delivery system of claim 6 wherein the at least one basic stabilizing additive is selected from the group consisting of magnesium carbonate, magnesium hydroxide, magnesium oxide, magnesium trisilicate, zinc carbonate, zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, and magaldrate.

8. The delivery system of claim 1 further comprising pore-forming agents that facilitate the escape of water-soluble acids produced by hydrolysis of the polymer.

9. The delivery system of claim 1 further comprising at least one stabilizing mono-, di-, or polysaccharide.

10. The delivery system of claim 9 wherein the at least one stabilizing mono-, di- or polysaccharide is selected from the group consisting of sorbitol, trehalose, and sucrose.

11. The delivery system of claim 1 wherein the form of the delivery system is injectable.

12. The delivery system of claim 11 wherein the injectable delivery system comprises reservoir-type or monolithic injectable vaccine antigen-containing particulates of injectable geometry.

13. The delivery system of claim 12 wherein the injectable delivery system is selected from the group consisting of microspheres, nanospheres, microcapsules, and cylindrical rods.

14. The delivery system of claim 1 wherein the form of the delivery system is an in-situ forming polymer.

15. The delivery system of claim 1 wherein the at least one formaldehyde-treated vaccine antigen is tetanus toxoid, diphtheria toxoid, or both tetanus toxoid and diphtheria toxoid.

16. The delivery system of claim 1 wherein the biocompatible polymer matrix is non-degradable.

17. A delivery system for sustained release of formaldehyde-treated vaccine antigens in a biocompatible polymeric matrix, said system comprising:
a biocompatible polymer matrix selected from homo-polymers and co-polymers derived from lactic and glycolic acids or lactides and glycolides,
at least one formaldehyde-treated vaccine antigen;
at least one formaldehyde-interacting stabilizing agent to stabilize the at least one formaldehyde-treated vaccine antigen and to inhibit the formation of aggregates wherein the at least one stabilizing agent is selected from the group of formaldehyde-interacting amino acids consisting of lysine, histidine, arginine, and glutamine;
at least one basic stabilizing additive selected from the group consisting of magnesium carbonate, magnesium hydroxide, magnesium oxide, magnesium trisilicate, zinc carbonate, zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, and magaldrate; and
optionally at least one stabilizing mono-, di-, or polysaccharide selected from the group consisting of sorbitol, trehalose, and sucrose, wherein the at least one stabilizing agent, the at least one formaldehyde-treated vaccine antigen, the at least one basic stabilizing additive, and optionally the at least one stabilizing saccharide are encapsulated within the biocompatible polymer matrix.

18. A delivery system for sustained release of formaldehyde-treated vaccine antigens in a biocompatible polymeric matrix, said system comprising:
a biocompatible polymer matrix;
at least one formaldehyde-treated vaccine antigen; and
at least one α-amino-containing stabilizing agent to stabilize the at least one formaldehyde-treated vaccine antigen and to inhibit the formation of aggregates, wherein the at least one stabilizing agent and the at least one formaldehyde-treated vaccine antigen are encapsulated within the biocompatible polymer matrix.

19. A method for use with the delivery system of claim 1, said method comprising:
encapsulating at least one formaldehyde-treated vaccine antigen and at least one organic formaldehyde-interacting stabilizing agent in a biocompatible polymer matrix; and
solidifying the biocompatible polymer matrix, the at least one formaldehyde-treated vaccine antigen and the at least one stabilizing agent to provide a biocompatible polymeric system for controlled release of formaldehyde-treated vaccine antigens.

20. The method of claim 19 wherein the at least one stabilizing agent inhibits the formaldehyde-mediated aggregation of formaldehyde treated bovine serum albumin in the bovine serum albumen solubility assay by greater than 60%.

21. The method of claim 19 wherein the at least one stabilizing agent is selected from the group of formaldehyde-interacting amino acids consisting of lysine, histidine, arginine, and glutamine.

22. The method of claim 19 wherein the biocompatible polymer matrix is biodegradable.

23. The method of claim 22 wherein the biodegradable polymer matrix is selected from homo-polymers and co-polymers derived from lactic and glycolic acids or lactides and glycolides.

24. The method of claim 19 further comprising at least one basic stabilizing additive.

25. The method of claim 24 wherein the at least one basic stabilizing additive is selected from the group consisting of magnesium carbonate, magnesium hydroxide, magnesium oxide, magnesium trisilicate, zinc carbonate, zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, and magaldrate.

26. The method of claim 19 further comprising pore-forming agents that facilitate the escape of water-soluble acids produced by hydrolysis of the polymer.

27. The method of claim 19 further comprising at least one stabilizing mono-, di-, or polysaccharide.

28. The method of claim 27, wherein the stabilizing mono, di-, or polysaccharide is selected from the group consisting of sorbitol, trehalose, and sucrose.

29. The method of claim 19 wherein the method forms an injectable delivery system.

30. The method of claim 29, wherein the injectable delivery system comprises reservoir-type or monolithic injectable vaccine antigen-containing particulates of injectable geometry.

31. The method of claim 30, wherein the injectable delivery system of is selected from the group consisting of microspheres, nanospheres, microcapsules, and cylindrical rods.

32. The method of claim 19 wherein the method forms an in-situ forming polymer.

33. The method of claim 19 wherein the at least one formaldehyde-treated vaccine antigen is tetanus toxoid, diphtheria toxoid, or both tetanus toxoid and diphtheria toxoid.

34. The method of claim 19 wherein the biocompatible polymer matrix is non-degradable.

35. A method for use with the delivery system of claim 1, said method comprising:
forming a biocompatible polymer matrix selected from homo-polymers and co-polymers derived from lactic and glycolic acids or lactides and glycolides;
encapsulating in the biocompatible polymer matrix,
at least one formaldehyde-treated vaccine antigen;
at least one stabilizing agent selected from the group of formaldehyde-interacting amino acids consisting of lysine, histidine, arginine, and glutamine;
at least one basic stabilizing additive selected from the group consisting of magnesium carbonate, magnesium hydroxide, magnesium oxide, magnesium trisilicate, zinc carbonate, zinc hydroxide, zinc phosphate, aluminum hydroxide, basic aluminum carbonate, dihydroxyaluminum sodium carbonate, dihydroxyaluminum aminoacetate, ammonium phosphate, calcium phosphate, calcium hydroxide, and magaldrate;
optionally at least one stabilizing mono, di-, or polysaccharide selected from the group consisting of sorbitol, trehalose, and sucrose, and
solidifying the biocompatible polymer matrix to provide a biocompatible polymeric system for controlled release of formaldehyde-treated vaccine antigens.
